(19) United States
(12) Patent Application Publication
(10) Pub. No.: US 2004/0097397 A1
(43) Pub. Date: May 20, 2004

(54) Perfume composition with enhanced viscosity and process for their preparation

(76) Inventors: Bernhard Mohr, Heidelberg (DE); Werner Bertleff, Viernheim (DE); Johan Smets, Lubbeek (BE); Jean Wevers, Steenhuffel (BE)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/380,013

(22) PCT Filed: Dec. 20, 2000
(86) PCT No.: PCT/EP00/13004

(30) Foreign Application Priority Data
Dec. 22, 1999 (EP) 99870277.3
Apr. 13, 2000 (EP) 00870070.0
Jun. 22, 2000 (DE) 200 02 168.1

(51) Int. Cl. A61K 7/46

(52) U.S. Cl. 512/1

(57) ABSTRACT

Perfume compositions that have enhanced viscosity and a process for making same are disclosed. Compositions containing such perfume compositions as well as methods of making and using same are also disclosed.

### DESCRIPTION

### TECHNICAL FIELD

The invention relates to a perfume composition containing at least one perfume compound and at least one polyamine, to a process of its production and its use in laundry, cleaning or fabric care compositions and in softeners

### BACKGROUND OF THE INVENTION

Laundry and cleaning products are well-known in the art. However, consumer acceptance of laundry and cleaning products is determined not only by the performance achieved with these products but also by the aesthetics associated therewith. The perfume components are therefore an important aspect of the successful formulation of such commercial products.

It is also desired by consumers for laundered fabrics to maintain the pleasant fragrance. Indeed, perfume additives make laundry compositions more aesthetically pleasant to the consumer, and in some cases the perfuse imparts a pleasant fragrance to fabrics treated therewith. However, the amount of perfume deposited onto fabrics from an aqueous laundry bath is often marginal and does not last long on the fabric. Furthermore, fragrance materials are often very costly and their inefficient use in laundry and cleaning compositions and ineffective delivery to fabrics results in a very high cost to both consumers and laundry and cleaning manufacturers. Industry, therefore, continues to seek with urgency for more efficient and effective fragrance delivery in laundry and cleaning products, especially for improvement in the provision of long-lasting fragrance to the fabrics. One solution is to use carrier mechanisms for perfume delivery. Such as by encapsulation, cf. U.S. Pat. No. 5,188,753.

Still another solution is to formulate compounds which provide a delayed release of perfume over a longer period of time than by the use of the perfume itself, cf. WO-A-95/04809, WO-A-95/08976 and co-pending European application No. 95303762.9. EP-A-0 971 026 relates to specific reaction products of an amino functional polymer comprising at least one primary amino group with an active aldehyde or ketone. Such compounds provide delayed release of the active ingredient such as a perfume. However, not-withstanding the advances in the art, there is still a need for a compound which provides a delayed release of the perfume component.

It is therefore the object of the invention to provide perfume compositions having an enhanced deposition of perfume on surfaces treated therewith and a delayed release of perfume.

### SUMMARY OF THE INVENTION

The object of the invention is achieved with a perfume composition which is obtainable by adding to 100 parts by weight of a mixture of

(a) 10 to 95% by weight of at least one perfume compound and

(b) 5 to 90% by weight of at least one polyamine,

the sum of (a) and (b) being always 100%, 0.1 to 20 parts by weight of at least one crosslinking agent having at least two groups which react with primary or secondary amino groups of the polyamine and crosslinking the mixture, and/or adding 0.1 to 30 parts by weight of a thickening agent.

The invention also relates to compositions containing the perfume composition of the invention and additional ingredients. These can be any type of composition which requires a perfume. Preferred compositions include laundry compositions and/or cleaning composition.

Still in another aspect of the invention, there is provided the use of the perfume composition of the invention for the manufacture of a laundry and cleaning composition for delivering residual fragrance onto the fabrics on which it is applied, typically by contacting a surface with the composition. “Contacting” is defined as “intimate contact of a surface with an aqueous solution of the herein above described composition.” Contacting typically occurs by soaking, washing, rinsing the composition onto fabric, but can also include contact of a substrate inter alia a material onto which the composition has been absorbed, with the fabric.

Laundry compositions also encompass compositions providing colour care, softening, anti-wrinkling care, composition counteracting malodours, as well as compositions suitable for use in any steps of the domestic treatment, that is as a pre-treatment composition, as a wash additive as a composition suitable for use in the rinse-cycle of the laundry cycle or applied on a dryer-sheet. Obviously, multiple applications can be wade such as creating the fabric with a pre-treatment composition of the invention and also thereafter with a composition of the invention suitable for use in the rinse cycle and/or suitable for use as a dryer-sheet.

The compositions can be solid, including in particular tablets and granular formulations, or liquid, including aqueous and non-aqueous liquids, including also liquids in the from a spray, foam, or aerosol form which for example can be suitable for use while ironing, or applied on the surfaces of the tumble dryer.

Most preferred herein are laundry detergent compositions and fabric care compositions such as softening compositions including rinse added softening composition as well as dryer added softening compositions.

Still in another aspect of the invention, there is provided a packaged composition comprising the processed product of the invention or composition of the invention. Preferably, the packaged composition is a closed packaging system having a moisture vapour transmission rate of less than 20 g/m²/24 hours. Typical disclosure of such a package can be found in WO-A-98/40464.

Still another preferred package is a spray dispenser, to create an article of manufacture that can facilitate treatment of fabric articles and/or surfaces with said compositions containing the perfume composition herein and other ingredients (examples are cyclodextrins, polysaccharides, polymers, surfactant, perfume, softener) at a level that is effective, yet if not discernable when dried on the surfaces. The spray dispenser comprises manually activated a non-manual powered (operated) spray means and a container containing the treating composition. Typical disclosure of such spray dispenser can be found in WO-A-96/04940 page 19 line 21 to page 22 line 27. The articles of manufacture preferably are in association with instructions for use to ensure that the consumer applies sufficient ingredient of the composition to provide the desired benefit. Typical compositions to be dispensed from a sprayer contain a level of amine reaction product of from out 0.01% to about 5%, preferably from about 0.05% to about 2%, more preferably from about 0.1% to out 1%, by weight of the usage composition.

A conventional disclosure of softening ingredients to be used in the softening composition of the invention can be found in EP-A-0971021, incorporated herein by reference, which typically include components selected from a surfactant like a quaternary ammonium softening component, a stabilising agent like a nonionic ethoxylated surfactant, a chelating agent, a crystal growth inhibitor, soil release agent, a polyalkyleneimine component, brighteners, preservatives, antibacterials, cyclodextrins, and mixtures thereof.

A conventional disclosure of a laundry or cleaning composition can be found in BP-A-0,659,876 and EP-A-0971021 which are both incorporated herein by reference.

Typical laundry or cleaning composition comprises a detergent and/or cleaning ingredient. By detergent or cleaning ingredient, it is meant ingredient which are respectively conventional to the detergent composition or cleaning composition. Typical ingredients of detergent compositions include one or more of surfactants, and organic and/or inorganic builders. The preferred laundry or cleaning composition will also preferably contain a bleaching system and/or other components conventional in detergent compositions. Typical of bleaching systems include a peroxyacid, or a bleach precursor, for example a peroxyacid precursor with a source of alkaline hydrogen peroxide necessary to form a peroxyacid bleaching species in the wash solution. Other optionals include soil suspending and anti-redeposition agents, suds suppressors, enzymes, fluorescent whitening agents, photoactivated bleaches, colours and additional perfume, and mixtures thereof.

In addition, when the composition is a laundry composition, it is preferred that the detergent composition comprises a softening clay.

The compositions of the invention (i.e. laundry, cleaning and fabric care compositions or softeners) may preferably contain a clay, preferably present at a level of from 0.05% to 40%, more preferably from 0.5% to 30%, most preferably from 2% to 20% by weight of the composition. For clarity, it is noted that the term clay mineral compound, as used herein, excludes sodium aluminosilicate zeolite builder compounds, which however, may be included in the compositions of the invention as optional components.

One preferred clay may be a bentonite clay. Highly preferred are smectite clays, as for example disclosed in the U.S. Pat. Nos. 3,862,058 3,948,790, 3,954,632 and 4,062,647 and European Patents No.s EP-A-299,575 and EPA-313,146 all in the name of the Procter and Gamble Company.

The term smectite clays herein includes both the clays in which aluminium oxide is present in a silicate lattice and the clays in which magnesium oxide is present in a silicate lattice. Smectite clays tend to adopt an expandable three layer structure.

Specific examples of suitable smectite clays include those selected from the classes of the montmorillonites, hectorites, volchonskoites, nontronites, saponites and sauconites, particularly those having an alkali or alkaline earth metal ion within the crystal lattice structure. Sodium or calcium montmorillonite are particularly preferred.

Suitable smectite clays, particularly montmorillonites, are sold by various suppliers including English China Clays, Laviosa, Georgia Kaolin and Colin Stewart Minerals.

Clays for use herein preferably have a particle dimension of from 10 nm to 800 nm more preferably from 20 nm to 500 nm, most preferably from 50 nm to 200 nm.

Particles of the clay mineral compound may be included as components of agglomerate particles containing other detergent compounds. Where present as such components, the term “largest particle dimension” of the clay mineral compound refers to the largest dimension of the clay mineral component as such, and not to the agglomerated particle as a whole.

Substitution of small cations, such as protons, sodium ions, potassium ions, magnesium ions and calcium ions, and of certain organic molecules including those having positively charged functional groups can typically tae place within the crystal lattice structure of the smectite clays. A clay may be chosen for its ability to preferentially absorb one cation type, such ability being assessed by measurements of relative ion exchange capacity. The smectite clays suitable herein typically have a cation exchange capacity of at least 50 meq/100 g. U.S. Pat. No. 3,954,632 describes a method for measurement of cation exchange capacity.

The crystal lattice structure of the clay mineral compounds may have, in a preferred execution, a cationic fabric softening agent substituted therein. Such substituted clays have been termed hydrophobically activated, clays. The cationic fabric softening agents are typically present at a weight ratio, cationic fabric softening agent to clay, of from 1:200 to 1:10, preferably from 1:100 to 1:20. Suitable cationic fabric softening agents include the water insoluble tertiary amines or dilong chain amide materials as disclosed in GB-A-1 514 276 and EP-B-0 011 340.

A preferred commercially available “hydrophobically activated” clay is a bentonite clay containing approximately 40% by weight of a dimethyl ditallow quaternary ammonium salt sold under the tradename Claytone EM by English China Clays International.

The compositions of the invention may—in addition to the perfume composition—contain a clay flocculating agent, preferably present at a level of from 0.005% to 10%, more preferably from 0.05% to 5%, most preferably from 0.1% to 2% by weight of the composition.

The clay flocculating agent functions such as to bring together the particles of clay compound in the wash solution and hence to aid their deposition onto the surface of the fabrics in the wash. This functional requirement is hence different from that of clay dispersant compound which are commonly added to laundry detergent compositions to aid the removal of clay soils from fabrics and enable their dispersion within the wash solution.

Preferred as clay flocculating agents herein are organic polymeric materials having an average weight of from 100,000 to 10,000,000, preferably from 150,000 to 5,000,000, more preferably from 200,000 to 2,000,000.

Suitable organic polymeric materials comprise homopolymers or copolymers containing monomeric units selected from alkylene oxide, particularly ethylene oxide, acrylamide, acrylic acid, vinyl alcohol, vinyl pyrrolidone, and ethylene imine. Homopolymers of, on particular, ethylene oxide, but also acrylamide and acrylic acid are preferred.

European Patents No.s EP-A-329,575 and EP-A-313,146 in the name of the Procter and Gamble Company describe preferred organic polymeric clay flocculating agents for use herein.

The weight ratio of clay to the flocculating polymer is preferably from 1000:1, to 1:1, more preferably from 500:1 to 1:1, most preferably from 300:1 to 1:1, or even more preferably from 80:1 to 10:1, or in certain applications even from 60:1 to 20:1.

In one embodiment of the invention, an effervescence source is present, preferably comprising an organic acid, such as carboxylic acids or aminoacids, and a carbonate. Then it may be preferred that part or all of the carbonate salt herein is premixed with the organic acid, and thus present in an separate granular component.

Effervescent means may also be optionally used in the compositions of the invention.

Effervescency as defined herein means the evolution of bubbles of gas from a liquid, as the result of a chemical reaction between a soluble acid source and an alkali metal carbonate, to produce carbon dioxide gas,

i.e. C₆H₈O₇+3NaHCO₃→Na₃C₆H₅O₇+3CO₂↑+3H₂O

Further examples of acid and carbonate sources and other effervescent systems may be found in: (Pharmaceutical Dosage Forms: Tablets Volume 1 Page 287 to 291).

Suitable alkali and/or earth alkali inorganic carbonate salts herein include carbonate and hydrogen carbonate of potassium, lithium, sodium, and the like amongst which sodium and potassium carbonate are preferred. Suitable bicarbonates to be used herein include any alkali metal salt of bicarbonate like lithium, sodium, potassium and the like, amongst which sodium and potassium bicarbonate are preferred. However, the choice of carbonate or bicarbonate or mixtures thereof may be made depending on the DE desired in the aqueous medium wherein the granules are dissolved. For example where a relative high pH is desired in the aqueous medium (e.g. above pH 9.5) it may be preferred to use carbonate alone or to use a combination of carbonate and bicarbonate wherein the level of carbonate is higher than the level of bicarbonate. The inorganic alkali and/or earth alkali carbonate salt of the compositions of the invention comprises preferably a potassium or more preferably a sodium salt of carbonate and/or bicarbonate. Preferably, the carbonate salt comprises sodium carbonate, optionally also a sodium bicarbonate.

The carbonate may have any particle size. In one embodiment, in particular when the carbonate salt is present in a granule and not as separately added compound, the carbonate salt has preferably a volume median particle size from 5 to 375 micrometers, whereby preferably at least 60%, preferably at least 70% or even at least 80% or even at least 90% by volume, has a particle size of from 1 to 425 micrometers. More preferably, the carbon dioxide source has a volume median particle size of 10 to 250, whereby preferably at least 60%, or even at least 70% or even at least 80% or even at least 90% by volume, has a particle size of from 1 to 375 micrometers; or even preferably a volume median particle size from 10 to 200 micrometers, whereby preferably at least 60%, preferably at least 70% or even at least 80% or even at least 90% by volume, has a particle size of from 1 to 250 micrometers.

In particular when the carbonate salt is added as separate component, so to say ‘dry-added’ or admixed to the other detergent ingredients, the carbonate may have any particle size, including the above specified particle sizes, but preferably at least an volume average particle size of 200 micrometers or even 250 micrometers or even 300 micrometers.

The inorganic carbonate salts herein are preferably present at a level of at least 20% by weight of the composition. Preferably they are present at a level of at least 23% or even 25% or even 30% by weight, preferably up to about 60% by weight or more preferably up to 55% or even 50% by weight.

They may be added completely or partially as separate powdered or granular component, as co-granules with other detergent ingredients, for example other salts or surfactants. In solid detergent compositions of the invention, they may also completely or partially be present in detergent granules such as agglomerates or spray dried granules.

Preferred effervescent source are selected from compressed particles of acid and carbonate optionally with a binder; and particle of carbonate, bicarbonate and citric acid or malic or maleic acid, preferably in weight ratios of 4:2:4.

The following granular or tablet laundry detergent formulations are in accord with the invention (in % by weight).

TABLE 1

| | 1 | 2 | 3 | 4 | 5 | 6 |
|---|---|---|---|---|---|---|
| Blown powder | | | | | | |
| Anionic surfactant | 23.0 | 20.0 | 12.0 | 17.0 | 10.0 | 7.0 |
| Nonionic surfactant | — | — | — | — | 2.0 | 4.0 |
| Zeolite A or | 10.0 | 18.0 | 14.0 | 12.0 | 10.0 | 10.0 |
| phosphate | | | | | | |
| polycarboxylate | 3.0 | 0.5 | 2.0 | 1.0 | 5.0 | 2.0 |
| polymer | | | | | | |
| Sulfate | — | 6.3 | 14.3 | 11.0 | 15.0 | 19.3 |
| Silicate (amorphous, | 10.0 | 10.0 | 1.0 | 6.0 | — | 8.0 |
| crystalline, | | | | | | |
| layered) | | | | | | |
| Carbonate | 13.0 | 19.0 | 8.0 | 20.0 | 8.0 | 6.0 |
| PEG 4000 | 0.4 | 1.5 | 1.5 | 1.0 | 1.0 | 1.0 |
| Chelant | — | 0.9 | 0.5 | — | — | 0.5 |
| Brightener | 0.3 | 0.2 | 0.3 | — | 0.1 | 0.3 |
| Perfume composition | 0.8 | 0.3 | 0.3 | 2.0 | 0.3 | 1.0 |
| Agglomerates: | | | | | | |
| Anionic surfactant | — | 7.0 | 7.0 | 2.0 | — | 7.0 |
| Zeolite A/phosphate | — | 7.5 | 7.5 | 8.0 | — | 7.5 |
| Carbonate | — | 4.0 | 4.0 | 5.0 | — | 4.0 |
| PEG 4000 | — | 0.5 | 0.5 | — | — | 0.5 |
| Misc (water etc) | — | 2.0 | 2.0 | 2.0 | — | 2.0 |
| Dry additives: | | | | | | |
| Citric acid | 5.0 | — | — | 5.0 | 2.0 | — |
| Percarbonate | 17.0 | — | — | — | 12.0 | 10.0 |
| Carbonate | 5.0 | 5.3 | — | 5.0 | 4.0 | 4.0 |
| Bleach activator | 4.0 | — | — | — | 8.0 | 3.6 |
| Enzyme(s) | 0.5 | 0.9 | 1.6 | 0.5 | 3.2 | 0.4 |
| Dye transfer | — | — | 0.5 | 0.3 | 0.5 | 0.1 |
| inhibitor | | | | | | |
| Silicone antifoam | 0.2 | 0.4 | 0.2 | 0.4 | 0.1 | — |
| Smectite clay | — | 9.0 | 5.2 | — | 10. | 3.0 |
| Misc/minors to 100% | | | | | | |

The following are high density liquid detergent compositions according to the present invention (in % by weight)

TABLE 2

| Ingredient | 7 | 8 | 9 | 10 |
|---|---|---|---|---|
| Polyhydroxy Coco-Fatty Acid | 2.50 | 2.50 | — | — |
| Amide | | | | |
| Nonionic ethoxylated alcohol | — | — | 10.0 | 10.0 |
| surfactant | | | | |
| Anionic surfactant | 20.0 | 20.0 | 16.0 | 2.0 |
| Alkyl N-Methyl Glucose Amide | — | — | 4.50 | 4.50 |
| Citric acid | 2.44 | 3.00 | 3.00 | 3.00 |
| Fatty acid | — | — | 2.00 | 2.00 |
| Ethanol | 3.00 | 2.81 | 3.40 | 3.40 |
| Monoethanolamine | 1.50 | 0.75 | 1.00 | 1.00 |
| Propanediol | 8.00 | 7.50 | 7.50 | 7.00 |
| Boric Acid/Borate | 3.50 | 3.50 | 3.50 | 3.50 |
| Ethoxylated | 0.50 | — | — | — |
| tetraethylenepentamine | | | | |
| Tetraethylenepentamine | — | 1.18 | — | — |
| Sodium Toluene Sulfonate | 2.50 | 2.25 | 2.50 | 2.50 |
| NaOH | 2.08 | 2.43 | 2.62 | 2.62 |
| Enzyme | 1.8 | 1.1 | 2.5 | 3.0 |
| Chelant | 0.5 | 0.7 | 2.5 | 0.5 |
| Perfume composition | 0.5 | 0.4 | 0.3 | 1.2 |
| Water | balance | balance | balance | balance |

The following fabric softening compositions are in accordance with the present invention (in % by weight):

TABLE 3

| | 11 | 12 | 13 | 14 | 15 | 16 |
|---|---|---|---|---|---|---|
| Cationic softener | 5.0 | 3.0 | 13.0 | 15.0 | 4.5 | 18.0 |
| Fatty acid | 0.3 | — | 1.0 | — | — | — |
| HCl | 0.02 | 0.02 | 0.02 | 0.02 | 0.02 | 0.02 |
| PEG | — | — | 0.6 | 0.6 | — | 0.6 |
| Perfume composition | 1.0 | 0.7 | 4.0 | 3.0 | 1.0 | 1.8 |
| Silicone antifoam | 0.01 | 0.01 | 0.01 | 0.01 | 0.01 | 0.01 |
| Electrolyte | — | — | 600 | 1200 | — | 1200 |
| (ppm) | | | | | | |
| Dye (ppm) | 10 | 10 | 50 | 50 | 10 | 50 |

Water and minors to balance to 100%

The perfume compound is, for example, selected from the group consisting of from α-damascone, δ-damascone, iso-damascone, carvone, γ-Methyl-lonone, 2,4,4,7-tetramethyl-oct-6-en-3-one, benzyl acetone, β-damascone, damascone, methyl dihydrojasmonate, methyl cedrylone, and mixtures thereof. Other examples of perfume compounds are 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpa-n-hexyl cinnamic aldehyde, bucinal, lyral, cymal, methyl nonyl acetaldehyde, hexanal trans-2-hexenal, and mixtures thereof.

The polyamine of the perfume composition is, for example, selected from the group consisting of polymers containing vinylamine units, polyethyleneimines, polymers grafted with ethylenelimine, polyallylamines, condensation products of piperazine, 1-(2-amino-ethyl)piperazine, 1,4-bis (3-aminopropyl)piperazine and mixtures thereof with crosslinkers, polymers containing lysine units, dendrimers containing primary amino groups, and mixtures thereof.

Preferred polyamines are polyethyleneimines having a molecular weight M_{w} of from 600 to 200,000.

Examples of crosslinking agents are epichlorohydrin, bischlorohydrin ethers of commands selected from the group consisting of ethylene glycol, polyethylene glycol having 2 to 100 glycol units, propylene glycols polypropylene glycols, copolymers of ethylene oxide and propylene oxide, glycerol, diglycerol, polyglycerol having up to 8 glycerol units, pentaerythritol and sorbitol, epoxides obtained from said bischlorohydrin ethers and mixtures thereof. Preferred crosslinking agents are diglycidyl ethers.

A preferred perfume composition contains, as thickener, an inorganic compound selected from the group consisting of hydrogenated castor oil, fumed silica or bentonite.

### DETAILED DESCRIPTION OF THE INVENTION

Perfume Composition

A typical disclosure of suitable fragrances traditionally used in perfumery can be found in “Perfume and Flavor Chemicals”, Vol. 1 and II, S. Arctander, Allured Publishing, 1994, ISBN 0-931710-35-5 and EP-A-0 971 026 (incorporated herein by reference). In the following list of perfume ingredients some compounds are commercial names known to those skilled in the art. These names include isomers which can also be used.

Perfume compositions are typically comprised of one or a mixture of perfume ingredients. One typical perfume ingredient is an aldehyde perfume ingredient. Preferably, the perfume aldehyde is selected from adoxal; anisic aldehyde; cymal; ethyl vanillin; florhydral; helional; heliotropin; hydroxycitronellal; koavone; lauric aldehyde; lyral; methyl nonyl acetaldehyde; P. T. bucinal; phenyl acetaldehyde; undecylenic aldehyde, vanillin; 2,6,10-trimethyl-9-undecenal, 3-dodecen-1-al, alpha-n-amyl cinnamic aldehyde, 4-methoxybenzaldehyde, benzaldehyde, 3-(4-tart butylphenyl)-propanal, 2-methyl-3-(para-methoxyphenyl propanal, 2-methyl-4-(2,6,6-trimethyl-2 (1)-cyclohexen-1-yl)butanal, 3-phenyl-2-propanal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclo-hexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal. octahydro-4,7-methano-1H-indenecarboxaldehyde, 3-ethoxy-4-hydroxybenzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, Undecenal, 2,4,6-trimethyl-3-cyclohexane-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cyclohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tert-butyl) propanal, dihydrocinnamic alaehyde, 1-methyl-4-(4-methyl-3-pentenyl)-3-cyclohexane-1-carboxaldehyde, 5 or 6 methoxy0hexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclhexenecarboxaldehyde, 7-hydroxy-3,7-dimethyl-octanal, trans-4-deceal, 2,6-nonadianal, para-tolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-mothanoindan-1-carboxaldehyde, 2-methyl octanal, alpha-methyl-4-(1-methyl ethyl)benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, Hexahydro-8,8-diethyl-2-naphthaldehyde, 3-propyl-bicyclo[2.2.1]-hept-5-ene-2-carbaldehyde, 9-decenal, 3-methyl-5-phenyl-1-pentanal, methylnonyl acetaldehyde, 1-p-menthene-q-carboxaldehyde, citral, lilial, florhydral, mefloral, and mixtures thereof.

More preferred aldehydes are selected from citral, 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2.6-nonadienal; alpha-n-amyl cinnamic aldehyde, alpha-n-hexyl cinnamic aldehyde, P.T. bucinal, lyral, cymal, methyl nonyl acetaldehyde, trans-2-nonenal, lilial, trans-2-nonenal, lauric aldehyde, undecylenic aldehyde, mefloral and mixture thereof.

Another typical perfume ingredient is a ketone perfume ingredient. Preferably, the perfume ketone is selected from buccoxime; is jasmone; methyl beta naphlthyl ketone; musk indanone; tonalid/musk plus; α-damascone, β-damascone, δ-amascone, iso-damascone, damascenone, damarose, methyl-dihydrojasmonate, menthone, carvone, camphor, fenchone, α-lonone, β-lonone, γ-methyl so-called lonone, flouramone, dihydrojasmome, cis-jasmone, iso-E-Super, methyl-cedrenyl-ketone or methyl-cedrylone, acetophenone, methyl-acetophenone, para-methoxy-acetophenone, methyl-β-naphtyl-ketone, benzyl-acetone, benzophenone, para-hydroxy-phonyl-butanone, celery ketone or Livescone, 6-lsopropyldecahydro-2-naphtone, dimethyl-octenone, freskomenthe, 4-(1-ethoxyvinyl)-3,3,5,5,-tetramethyl-cyclohexanone, methyl-heptenone, 2-(2-(4-methyl-3-cyclohexen-1-yl)propyl)-cyclopentanone, 1-(p-menthen-6(2)-yl)-1-propanane, 4-(4-hydroxy-3-methoxyphenyl)-2-butanone, 2-acetyl-3,3-dimethyl-norbornane, 6,7-dihydro-1,1,2,3,3-pentamethyl-4(5H)-lndanone, 4-damascol, dulcinyl or cassione, gelsone, hexalon, Isocyclemone E, methyl cyclocitrone, methyl-lavender-ketone, orivon, para-tertiary-butyl-cyclohexanone, verdone, delphone, tusaone, neobutenone, plieatone, veloutone, 2,4,4,7-tetramethyl-oct-6-en-3-one, tetrameran, hedione, and mixtures thereof.

The perfume composition may also contain a mixture of perfume ingredients. Typical of these ingredients include fragrant substance or mixture of substances including natural (i.e. obtained by extraction of flowers, herbs, leaves, roots, barks, wood, blossoms or plants), artificial (i.e., a mixture of different nature oils or oil constituents) and synthetic (i.e. synthetically produced) odoriferous substances. Such materials are often accompanied by auxiliary materials, such as fixatives, extenders, stabilizers and solvents. These auxiliaries are also included within the meaning of perfume, as used herein. Typically, perfumes are complex mixtures of a plurality of organic compounds.

Suitable perfumes are, for example, disclosed in U.S. Pat. No. 5,500,138 (incorporated herein by reference).

Examples of perfume ingredients useful in the perfume compositions include, but are not limited to, amyl salicylate; hexyl salicylate; terpineol; 3,7-dimethyl-cis-2,6-octadien-t-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphanyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenyl-propene; methyl-2-n-hexyl-3-oxo-cyclopentane carboxylate; undecalactone.

Additional examples of fragrance materials include orange oil; lemon oil; grapefruit oil; bergamot oil clove oil; dodecalactone gamma; methyl-2-(2-pentyl-3-oxo-cyclopentayl)acetate; β-naphthol methylether; methyl-β-naphcylketone; coumarin; 4-tert-butylcyclohexyl acetate; α,α-dimethylphenethyl acetate; methylphenylcarbinyl acetate; cyclic ethyleneglycol diester of tridecandioic acid; 3,7-dimethyl-2,6-octadiene-1-nitrile; ionone gamma methyl; ionone alpha; ionone beta; petitgrain; methyl cedrylone; 7-acetyl-1,2,3,4,5,6,7,8-octahydro-1,1,6,7-tetramethyl-naphthalene; ionone methyl; methyl-1,6,10-trimethyl-2,5,9-cyclododecatrion-1-yl ketone; 7-acetyl-1,1,3,4,4,6-hexamethyl tetrilin; 4-acetyl-6-tert-butyl-1,1-dimethyl indane; benzophenone; 6-acetyl-1,1,2,3,3,5-hexamethyl indane; 5-acetyl-3-isopropyl-1,1,2,6-tetramethyl indane; 1-dodecanal; 7-hydroxy-3,7-dimethyl octanal; 10-undecen-1-al; iso-hexenyl cyclohexyl carboxaldehyde; formyl tricyclodecan; cyclopentadecanolide; 16-hydroxy-9-hexadecenoic acid lactone; 1,3,4,6,7,8-hexahydro-4,6,6,7,8,8-hexamethylcyclopenta-gamma-2-benzopyrane; ambroxane; dodecahydro-3a,6,6,9a-tetramethylnaphbtho-[2,1b]furan; cedrol; 5-(2,2,3-trimethylcyclopent-3-enyl)-3-methylpentan-2-ol; 2-ethyl-4-(2,2,3-trimethyl-3-cyclopenten-1-yl)-2-buten-1-ol; caryophyllene alcohol; cedryl acetate; para-tert-butylcyclohexyl acetate; patchoulil olibanum resinoid; labdanum; vetivert; copaiba balsam; fir balsam; hydroxycitronellal and indol; phenyl acetaldehyde and indol.

More examples of perfume components are geraniol; geranyl acetate; linalool; linalyl acetate; tetrahydrolinalool; citronellol; citronellyl acetate; dihydromyrcenol; dibydromyrcenyl acetate; tetrahydromyrcenol; terpinyl acetate; nopol; nopyl acetate; 2-phenylethanol; 2-phenylethyl acetate; benryl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetivteryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitriler cedryl acetal; 3-isocamphylcyclohexanol; cedryl methylether; isolongiflanone; aubepine nitrile; aubepine; heliotropine; eutenol; vanillin; diphenyl oxide; hydroxycitronellal ionones; methyl ionones; isomethyl ionomes; irones; cis-3-hexenol and esters thereof; indane musk fragrances; tetralin musk fragrances; isochroman musk fragrances; macrocyclic ketones; macrolactone musk fragrances; ethylene brassylate.

Also suitable herein as perfume ingredients of the perfume composition are the so-called Schiff bases. Schiff-bases are the condensation products of an aldehyde perfume ingredient with an anthranilate. A typical description can be found in U.S. Pat. No. 4,853,369. Typical Schiff bases are selected from the group consisting of 4-(4-hydroxy-4-methylpentyl)-3-cyclohexene-1-carboxaldehyde and methyl anthtanilate; condensation products of hydroxycitronellal and methyl anthranilate; condensation products of 4-(4-hydroxy-4-methyl pentyl)-3-cyclohexene-1-carboxaldehyde and methyl anthranilate; condensation products of methyl anthranilate and hydroxy citronellal (commercially available under the tradename Aurantiol); condensation products of methyl anthranilate and methyl nonyl acetaldehyde (commercially available under the tradename Agrumea); condensation products of methyl anthranilate and PT Bucinal (commercially available under the tradename Verdantiol); condensation products of methyl anthranilate and Lyral (commercially available under the tradename Lyrame); condensation products of methyl anthranilate and Ligustral (commercially available under the tradename Ligantral), and mixtures thereof.

Preferably, the perfume compositions useful in the present invention compositions are substantially free of halogenated materials and nitromusks.

More preferably, the perfume compounds are characterised by having a low Odor Detection Threshold. Such Odor Detection Threshold (ODT) should be lower than 1 ppm, preferably lower than 10 ppb—measured under controlled Gas Chromatography (GC) conditions such as described here below. This parameter refers to the value commonly used in the perfumery arts and which is the lowest concentration at which significant detection takes place that some odorous material is present. Please refer for example to “Compilation of Odor and Taste Threshold Value Data (ASTM DS 48 A)”, edited by F. A. Fatzalari, International Business Machines, Hopwell Junction, N.Y. and in Calkin et al., Perfumery, Practice and Principles, John Willey & Sons, Inc., page 243 et seq. (1994). For the purpose of the present invention, the Odor Detection Threshold is measured according to the following method:

The gas chromatograph is characterized to determine the exact volume of material injected by the syringe, the precise split ratio, and the hydrocarbon response using a hydrocarbon standard of known concentration and chainlength distribution. The air flow rate is accurately measured and, assuming the duration of a human inhalation to last 0.02 minutes, the sampled volume is calculated. Since the precise concentration at the detector at any point in time is known, the mass per volume inhaled is known and hence the concentration of material. To determine the ODT of a perfume material, solutions are delivered to the sniff port at the back-calculated concentration. A panelist sniffs the GC effluent and identifies the retention time when odor is noticed. The average over all panelists determines the threshold of noticeability. The necessary amount of analyte is injected onto the column to achieve a certain concentration, such as 10 ppb, at the detector. Typical gas chromatograph parameters for determining odor detection threshold are listed below.

GC: 5890 Series II with FID detector

7673 Autosampler

Column: J&W Scientific DB-1

Length 30 meters ID 0.25 mm film thickness 1 micrometer

Method:

Split Injection: 17/1 split ratio

Autosampler: 1.13 microliters per injection

Column Plow: 110 mL/minute

Air Flow: 345 mL/minute

Inlet Temp. 245° C.

Detector Temp. 280° C.

Temperature Information

Initial Temperature: 50° C.

Rate: 5 C/minute

Final Temperature: 280° C.

Final Timer 6 minutes

Leading assumptions: 0.02 minutes per sniff

GC air adds to sample dilution

Examples of such preferred perfume components are those selected from: 2-methyl-2-(para-iso-propylphenyl)-propionaldehyde, 1-(2,6,6-trimethyl-2-cyclohexan-1-yl)-2-buten-1-one and/or paramethoxy-acetophenone. Even more preferred are the following compounds having an ODT of at least 10 ppb measured with the method described above: undecylenic aldehyde, undecalactone gamma, heliotropin, dodecalactone gamma, p-anisic aldehyde, para hydroxy-phenyl-butanone, cymal, benzyl acetone, ionone alpha, p.t.bucinal, damascenone, ionone beta, methyl-nonyl ketone, methyl heptine carbonate, linalool, indol, cis-3-hexenyl salicylate, vanillin, methyl isobutenyl tetrahydropyran, ethylvanillin, coumarin, ethyl methyl phenyl glycidate, eugenol, methylanthranilate, iso eugenol, beta naphtol methyl ester, herbavert, lyral, allyl amyl glycolate, dihydro iso jasmtonate, ethyl-2-methyl-butyrate, nerol, and phenylac taldehyde. Most preferably the perfume composition comprises at least 5%, more preferably at least 10% of such components.

Most preferably, the perfume ingredients are those as described in WO-A-96/12785 on pages 12-14. Even most preferred are those perfume compositions comprising at least 10%, preferably 25%, by weight of perfume ingredient with a ClogP of at least 2.0, preferably at least 3.0, and a boiling point of at least 250° C. Still another preferred perfume composition is a composition comprising at least 20%, preferably 35%, by weight of perfume ingredient with a ClogP of at least 2.0, preferably at least 3.0, and boiling point of less than or equal to 250° C.

ClogP is a commonly known calculated measure as defined in the following references “Calculating log P_{oct} from Structures”; Albert Leo (Medicinal Chemistry Project, Pomona Collage, Claremont, Calif., USA; Chemical Reviews, Vol. 93, number 4, June 1993; as well as from Comprehensive Medicinal Chemistry, Albert Leo, C. Hansch, Ed. Pergamon Press; Oxford, 1990, Vol. 4, p.315; and Calculation Procedures for molecular lipophilicity: a comparative Study, Quant. Struct. Act. Realt. 15, 403-409 (1996), Raymund Mannhold and Karl Dross.

Polyamines

Suitable polyamines may be selected from aminoaryl derivatives containing at least two primary or secondary amino groups, polyamines having at least two primary or secondary nitrogen atoms, polyamino acids and their derivatives, cross linked polyamino acids, glucamines, polyamidoamines, crosslinked polyamidoamines, amino substituted polyvinylalcohol, bis amine of polyalkylene glycols, bis aminopropyl-terminated polyalkylene glycols, poly[oxy(methyl-1,2-ethanediyl)], α-(2-aminomethylethyl)-ω-(2-aminomethyl-ethoxy (=C.A.S. No. 9046-10-0), poly[oxy(methyl-1,2-ethanediyl], α-hydro-)-(ω)-(2-amino-methylethoxy)-, ether with 2-ethyl-2-(hydroxymethyl)1,3-propandiol (=C.A.S. No. 39423-51-3); commercially available under ther tradename Jeffamines T-403, D-230, D-400, D2000; 2,2′,2″-triaminotriethylamine; 2,2′-diamino-diethylamine 3,3′-diaminodipropylamine, 1,3-bis aminoethyl-cyclohexane commercially available from Mitsubishi and the C₁₂-Sternamines commercially available from Clariant like the C₁₂-sternamin(propylenamine)ₙ with n=3/4 polyethylenimine dendrimers, polypropylenimine dendrimers, the commercially available Starburst polyamidoamines (PAMAM) dendrimers, generation GO-G10 from Dentritech, the dendrimers Astromols®, generation 1·5 from DSM being DiAminoButan PolyAmine DAB (PA)x dendrimers with x=2ₙ×4 and n being generally comprised between 0 and 4, polymers containing vinylamine units, polyethyleneimines, polymers grafted with ethyleneimine, polyallylamines, condensation products of piperazine, 1-(2-aminoethyl)piperazine. 1,4-bis(3-aminopropyl)piperazine and mixtures thereof with crosslinkers, polymers containing lysine units, and mixtures thereof.

Preferred polyamines are polymers consisting of or containing vinylamine units. The polymers belonging to this group are known for example from U.S. Pat. No. 4,421,602, U.S. Pat. No. 4,444,667, and U.S. Pat. No. 5,324,792 (all incorporated by reference). They are obtainable by homo or copolymerization of N-vinylformamide and hydrolysis of these copolymers with acids or bases or enzymatically. During hydrolysis the formyl group of the homopolymers of N-vinylformamide or of the copolymers of N-vinylformamide is cleaved under formation of a primary amino or ammonium group. If the hydrolysis is carried out with an acid such as sulfuric acid, hydrogen chloride or formic acid, then the polymer contains vinyl ammonium units, i.e. the salts of the acid used for hydrolysis. The N-vinylformamide unit in the polymers can be partially or completely hydrolyzed. The degree of hydrolysis can be 1 to too, preferably 5 to 100 or 10 to 95%. If a homopolymer of N-vinylformamide is hydrolyzed at a degree of 100%, the polymer obtained is polyvinylamine. If the hydrolysis in carried out partially, the polymer obtained contains N-vinylformamide units and vinylamine units depending of the degree of hydrolysis.

Polymers containing vinyl amine units are also obtainable from copolymers of N-vinylformamide with one or more comonomers and hydrolysis of the copolymers. The degree of hydrolysis of the polymerized N-vinylformamide may be the same as specified above for hydrolysis of the homopolymers of N-vinylformamide. Suitable comonomers are, for example, vinyl esters of saturated carboxylic acids of 1 to 6 carbon atoms, e.g. vinyl formiate, vinyl acetate, vinyl propionate and vinyl butyrate, esters of ethylenically unsaturated mono or dicarboxylic acids containing 3 to 6 carbon atoms, e.g. methyl acrylate, methyl methacrylate, ethyl acrylate, ethyl methacrylate, isopropyl acrylate, n-butyl acrylate, isobutyl acrylate, hydroxyethyl acrylate, hydroxybutyl acrylate, hydroxybutyl methacrylate and monoesters of acrylic and methacrylic acid with polyalkylene glycols having a molecular weight M_{w} from 200 to 10,000 preferably 400 to 2,000. Farther examples of suitable monomers are esters of the said acids with aminoalcohols such as dimethylamino ethyl acrylate, dimethylamino methycrylate, dimethylaminopropyl acrylate and dimethylaminopropyl methacrylate

Other suitable comonomers are unsaturated amides such as acrylamide, methacrylamide and N-alkylmonoamides and N-alkyldiamides having alkyl radicals of 1 to 6 carbon atoms, e.g. N-mathylacrylamide, N,N-dimethylacrylamide, N-methylmethacrylamide, N-ethylmethacrylarmide, N-isopropylacrylamide, N-n-propylacrylamide and basic acrylamides such as dimethylaminoethylacrylamide, dimethylaminomethacrylamide, dimethylaminopropylacrylamide and dimethylaminopropylmethacrylamide.

Other suitable comonomers are vinyl ethers having alkyl groups of from 1 to 18 carbon atoms, e.g. methyl vinyl ether, ethyl vinyl ether, n-propylvinyl ether, isopropyl vinyl ether, n-butyl vinyl ether, n-pentyl vinyl ether and n-hexyl vinyl ether, or vinyl ethers having aromatic substituents such as phenyl vinyl ether or benzyl vinyl ether.

Other suitable comonomers are N-vinyl pyrrolidone, N-vinyl caprolactam, acrylonitrile, methacrylonitrile, N-vinyllmidazole and substituted N-vinylimidazoles such as N-vinyl-2-methylimidazole, N-vinyl-4-methylimidazole and N-vinyl-2-ethylimidazole, N-vinylimidazo ins, N-vinyl-2-methylimidazoline and N-vinyl-2-ethylimidazoline. N-vinylimidazoles and N-vinylimidazolines are used not only in the form of the free bases but also in a form neutralized with mineral acids or with organic acids or in quaternized form, quaternization preferably being carried out with dimethylsulfate, diethylsulfate, methyl chloride or benzyl chloride.

The molar mass M_{w} of the polymers containing vinylamine units are, for example, form 1,000 to 10 million, preferably form 5,000 to 5 million (determined by light scattering). This molar mass corresponds, for example, to K values of from 5 to 300, preferably from 10 to 250 (determined according to H. Fikentscher in 5% strength by weight aqueous sodium chloride solution at 25° C. and at a polymer concentration of 0.5% by weight). The polymers containing vinylazine units are preferably used in salt-free form. Salt-free solutions of such polymers can be prepared, for example, from the salt-containing solutions which for instance are obtained by hydrolysis of N-vinylformamide units containing polymers with acids such as hydrogen chloride or sulfuric acid, with the aid of ultrafiltration through suitable membranes with separation limits of, for example, 1,000 to 500,000, preferably from 10,000 to 300,000 dalton.

If desired, the copolymers may also contain additionally polymerized monomer units having at least two ethylenically unsaturated double bonds. Such monomers are usually used in the copolymerization as crosslinking agents. Thus, N-vinylformamide or mixtures of N-vinylforamide with 1 to 99 mol % of other monoethylenically unsaturated monomers can be additionally copolymerized with at least one crosslinker in an amount of from 0 to 5 mol %.

The above polymers of N-vinylformamide are hydrolysed to form polymers containing vinylamine units. Preferred polymers of this group are homopolymers of vinylamines and hydrolyzed copolymers of N-vinylformamide and vinylacetate contacting vinyl amine units and vinyl alcohol units. The vinyl alcohol units are formed by hydrolysis from vinylacetate units contained in the polymer.

Other suitable polymers containing vinylamine units are obtainable from polymers containing N-vinylformamide grafted on poly-saccharides or polyalkylexne glycols. The N-vinylformamide grafted polymers are hydrolyzed under formation of vinylamine units containing polymers. The polymers belonging to this group are known for example from U.S. Pat. No. 5,334,287, U.S. Pat. No. 6,048,945 and U.S. Pat. No. 6,060,566 (all incorporated by reference). Usually per 100 parts by weight of starch, a polyalkylene glycol such as polyethylene glycol, polypropylene glycol or block polymers of ethylene and propylene glycol, or a polyvinylester are grafted with from 1 to 100, preferably from 5 to 95 parts by weight of N-vinylformamide and are then completely or partially hydrolyzed.

Other compounds which contain primary amino groups are polyethyleneimines. They are prepared, for example, by polymerizing ethyleneimine in aqueous solution in the presence of acid-eliminating compounds, acids or Lewis acids. Polyethyleneimines have, for example, molar masses M_{w} of up to 2 million, preferably 200 to 500,000. Polyethyleneimines having molar masses M_{w}, of from 500 to 100,000 are particularly preferably used. Water-soluble crosslinked polyethyleneimines which are obtainable by reacting polyethyleneimines with crosslinking agents such as epilorohydrin or bischlorohydrin ethers of polyalkylene glycols with from 2 to 100 ethylene oxide and/or propylene oxide units or blockpolymers containing blocks of units of ethylene oxide and propylene oxide are also suitable.

Suitable amino- and/or ammonium-containing polymers are furthermore polyamidoamines grafted with ethyleneimine. These polymers are obtained, for example, by first condensing dicarboxylic acids with polyamines and then grafting the polyamidoamines thus obtained with ethyleneimine. Suitable polyamidoamines are obtainable by reacting dicarboxylic acids of 4 to 10 carbon atoms with polyalkylene polyamines which contain from 3 to 10 basic nitrogen atoms in the molecule. Examples of suitable dicarboxylic acids are succinic acid, maleic acid, adipic acid, glutaric acid, sebacic acid and terephthalic acid. In the preparation of the polyamidoamines, it is also possible to use mixtures of dicarboxylic acids as well as mixtures of a plurality of polyalkylenepolyamines. Suitable polyalkylenepolymines are, for example, diethylenetriamine, triethylenetetramine, tetraethylenepentamine, dipropylenetriamine, tripropylenetetramine, dihexamethylenptriamine, aminopropylethylendiamine and bisaminopropylethylenediamine. For the preparation of the polyamidoamines, the dicarboxylic acids and polyalkylenepolyamines are heated to relatively high temperatures, for example, to temperatures of from 120 to 220° C., preferably from 130 to 180° C. The water formed in the condensation is usually removed from the system. In the condensation it is also possible to use lactones or lactams of carboxylic acids of 4 to 18, preferably 6 to 12 carbon atoms. For example, from 0.8 to 1.4 mol of polyalkylanepolyamine are used per mol of dicarboxylic acid. The polyamidoamines thus obtained are grafted with ethyleneomine using for example, per 100 parts by weight of polyamidoamine 1 to 50 parts by weight of ethyleneimine. The grafting of the ethyleneimine is carried out in the presence of acids or Lewis acids, such as sulfuric acid or boron trifluoride etherates, at, for instance, from 80 to 100° C. Polyamidoamines can be crosslinked before being grafted with ethyleneimine. Suitable crosslinking agents are, for example, epichlorohydrin, bischlorohydrinethers of polyalkyleneglycols and bisepoxides of chlorohydrinethers of polyalkyleneoxides. Compounds of this type are describes for example in DE-B-24 34 816.

Polyallylamines are also suitable cationic synthetic polymers having primary amino and/or ammonium groups. Polymers of this type are obtained by homopolymerization of allylamine, preferably in a form neutralized with acids or in quaternized form or by copolymerization of allylamine with other monoethylinically unsaturated monomers which are described above as comonomers for N-vinylformamide. The K values of these polymers is of from 30 to 300, preferably from 100 to 180 (determined according to H. Fikentscher in 5% strength by weight aqueous sodium chloride solution at 25° C. and at a polymer concentration of 0.5% by weight). At a pH of 4.5, they have, for example, a charge density of at least 4 meq/g of polyelectrolyte.

Other suitable cationic synthetic polymers having primary amino groups are polylysines. Such polymers are obtained by condensing lysine alone or together with other compounds cocondensable therewith, for example, compounds having at least one carboxyl group, carboxylic acid anhydrides, diketenes, amines, lactams, alcohols, alkoxylated alcohol and/or alkoxylated amines.

Compounds of this type are disclosed in copending U.S. patent application Ser. No. 09/13123 (incorporated by reference).

Further synthetic polymeric compounds containing primary amino groups are polymers containing aminoethyl acrylate units and polymers containing aminoethyl methacrylate units. Aminoethyl acrylate and/or aminoethyl methacrylate may be polymerized alone or in combination or together with other monoethylenically unsaturated monomers. The molecular weight M_{w} of the polymers is, for example, of from 1,000 to 5 million, preferably of from 5,000 to 500,000.

Other suitable cationic synthetic polymers having primary amino and/or ammonium groups are condensation products of piparazine, 1-(2-aminoethyl) piperazine, 1,4-bis (3-aminopropyl) piperazine and mixtures thereof with crosslinkers. The condensation reaction is carried out in an aqueous medium. Condensation products of this type are disclosed in U.S. Pat. No. 6,025,322 (incorporated by reference).

Crosslinking Agents

Suitable crosslinkers, which contain at least two functional groups, are for example α-, ω- or vicinal dichloroalkanes such as 1,2-dichloroethane, 1,2-dichloropropane, 1,3-di chloropropane, 1,4-dichlorobutane and 1,6-dichlorohexane. Farther suitable crosslinkers are glycidyl halides such as epichlorohydrin, bischlorohydrin ethers of polyols, polychlorohydrin ethers of polyols, bischlorohydrin ethers of polyalkylene glycols, chloroformic acid esters, phosgene and in particular, halogen-free crosslinkers.

Preferably used crosslinkers are epichlorohydrin, bischlorohydrin ethers of ethylene glycol, polyethylene glycol having 2 to 100, especially 2 to 50 ethylene glycol units, propylene glycols, polypropylene glycols, copolymers of ethylene oxide and propylene oxide, glycerol, diglycerol, polyglycerol having up to 8 glycerol units, pentaerythritol and sorbitol, and halogen-free crosslinkers which are at least bifunctional and preferably selected from a the group consisting of:

(1) ethylene carbonate, propylene carbonate and/or urea,

(2) monoethylenically unsaturated carboxylic acids and their esters, amides and anhydrides, at least dibasic saturated carboxylic acids or polycarboxylic acids and also the esters, amides and anhydrides which are in each case derived therefrom,

(3) reaction products of polyether diamines, alkylene diamines, polyalkylene polyamines, alkylene glycols or polyalkylene glycols, or their mixtures, with monoethylenically unsaturated carboxylic acids or esters, amides or anhydrides of monoethylenically unsaturated carboxylic acids, with the reaction products exhibiting at least two ethylenically unsaturated double bonds, or carboxamide, carboxyl or ester groups as functional groups,

(4) reaction products of dicarboxylic acid esters with ethylenimine, which products contain at least two aziridino groups.

(5) diepoxides, polyepoxides, α,ω-diisocyanates such as hexamethylene diisocyanate and polyisocyanates

and also mixtures of the said crosslinkers. These compounds are, for example, disclosed in copending U.S. application Ser. No. 09/13123 as crosslinkers.

Thickening Agents

Materials used as thickening agents are, for example, described in “Chemistry and Technology of Lubricants”, R. M. Mortimer and S. T. Orszulik, VCH Publishers, New York, 1992; WO-A-99/61571 (thickeners for nonaqueous dishwashing detergents), EP-A-0,596,209 (crosslinked castor oil derivatives for use as thickeners for oils) and “Additives for Coatings,” L. Bieleman, VCR Publishers, New York, 2000 (all incorporated herein by reference).

Examples for components useful for the thickening according to the present invention are polymeric thickening agents, organic thickeners, inorganic thickening agents or mixtures thereof.

Suitable polymeric thickening agents comprise polymethacrylates, olefin copolymers, hydrogenated styrene-diene copolymers, polyamides, polyurea, silicone waxes and mixtures thereof.

Suitable organic thickening agents are, for example, hydrogenated castor oils, overbased sulphonates, esterified sorbitols and mixtures thereof. In a preferred embodiment of the invention hydrogenated castor oils are employed. These castor oil derivatives may be further chemically modified, e.g. crosslinked or used in form of their amides, ethers or esters. The hydrogenated castor oils may be used a powdered material, as paste, in solution or in dispersed form. The temperature for miring the thickening agents (c) with components (a) and (b) is, for example, of from 0° C. to 120° C., preferably from 20° C. to 80° C.

Suitable inorganic thickening agents are, for example, fumed silica, bentonites, hydrophobic talcite, aluminium dioxide, titanium dioxide disilicates and mixtures thereof. Generally the thickener is selected from the, group consisting of hydrogenated castor oil, fumed silica, bentanite and mixtures thereof. In an especially preferred embodiment of the invention fumed silica is used as thickener.

The invention also relates to a process for the production of a perfume composition by adding to 100 parts by weight of a mixture of

(a) 10 to 95% by weight at least one perfume compound and

(b) 5 to 90% by weight of at least one polyamine,

the sum of (a) and (b) being always 100%,

(c) 0.1 to 20 parts by weight of at least one crosslinking agent having at least two groups which react with primary or secondary amino groups of the polyamine and crosslinking the mixture, and/or adding 0.1 to 30 parts by weight of a thickening agent.

The polyamine is preferably selected from the group consisting of polymers containing vinylamine units, polyethyleneimines, polymers grafted with ethyleneimine, polyallylamines, condensation products of piperazins, 1-(2-aminoethyl) pipierazine, 1,4-bis(3-aminopropyl) piperazine and mixtures thereof with crosslinkers, polymers containing lysine units, dendrimers containing primary and/or secondary amino groups, and mixtures thereof. Especially preferred polyamines are selected from the group consisting of polyvinylamine, a copolymer containing vinylamine units, their salts with inorganic or organic acids, and mixtures thereof.

The polyamines are preferably crosslinked with a crosslinking agent selected from the group consisting of epichlorohydrin, bischlorohydrin ethers of ethylene glycol, polyethylene glycols having 2 to 100 glycol units, propylene glycols, polypropylene glycols, copolymers of ethylene oxide and propylene oxide, glycerol, diglycerol, polyglycerol having up to 8 glycerol units, pentaerythritol and sorbitol, epoxides obtained from said bischlorohydrin ethers, and mixtures thereof. Especially preferred crosslinkers are diglycidyl thereof ethylene glycol or polyethylene glycol having 2 to 50 ethylene glycol units. Polyglycidyl ethers of polyvalent alcohols such as pentaerythritol, sorbitol, glycerol and polyglycerol can also be used with advantage as crosslinking agents.

In a preferred embodiment of the invention, the polyamine can react with suitable ketones and/or aldehydes forming Schiff base-type reaction products as described in EP-A-0,971,026. This reaction step can be carried out before, in sequence of or following the viscosity enhancement reaction according to step (c) of the process for the production of the perfume composition. For this reaction perfume ketones are preferred for their odor character. These perfume ketones are selected from buccoxime; iso jasmone; methyl beta naphthyl ketone; musk indanone; tonalid/musk plus; Alpha-Damascone, Beta-Damascone, Delta-Damascone, Iso-Damascone, Damascenone, Damarose, Methyl-Dihydrojasmonate, Menthone, Carvone, camphor, Fenchone, Alpha-lonone, Beta-lonone, Gamma-Methyl so-called lonone, Fleuramone, Dihydro-japmone, Cis-Jasmone, Iso-E-Super, Methyl-Cedrenyl-ketone or Methyl-cedrylone, acetophenone, methyl-acetophenone, para-methoxy-acetophenone, methylbeta-naphtyl-ketons, benzyl-acetone, benzophenone, para-hydroxyphenyl-butanone, Celery ketone or Livescone. 6-loopropyldecahydro-2-naphtone, dimethyl-octenones Freskomen-the, 4-(1-Ethoxyvinyl)-3,3,5,5,-tetra-methyl-cyclohexanone, methyl-heptenone, 2-(2-(4-methyl-3-cyclohexen-1-yl)propyl)-cyclopentanone, 1-(p-menthen-6 (2)-yl)-1-propanone, 4-(4-hydroxy-3-methoxyphenyl)-2-butanone, 2-acetyl-3,3-dimethyl-norboranes 6,7-di-hydro-1,1,2,3,3-pentamethyl-4 (5H)-Indanone, 4-Damascol, Dulcinyl or Cassione, celsone, Hexalon, Isocyclemone E, Methyl Cyclocitrone, Methyl-Lavender-Katone, orivon, para-tertiary-butyl-cyclohexanone, Verdons, Delphone, Muscone, neobutenne, Plicatone, Veloutone, 2,4,4,7-tetramethyl-oct-6-en-3-one, Tetrameran, undecalactone and gamma undecalactone.

From the above mentioned compounds the more preferred ketones are selected for their odor character from Alpha Damascone, Delta Damascone, Iso Damascone, Carvone, Gamma-Methyl-lonone, Iso-E-Super, 2,4,4,7-tetramethyl-oct-6-en-3-one, benzyl acetone, Beta Damascone, Damascenone, methyl dihydrojasmonate, methyl cedrylone, and mixtures thereof.

More preferred aldehydes are selected for their odor character from 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P. T. bucinal, lyral, cymal, methyl nonyl acstaldehyde, hexanal, trans-2-hexenal, and mixtures thereof.

The crosslinking of components (a) and (b) is usually carried out at a temperature of from 0 to 120° C., preferably of from 20 to 80° C. Per 100 parts by weight of a mixture of components (a) and (b) 0.1 to 20, preferably 0.5 to 10 parts by weight of at least one crosslinking agent are used. The perfume compositions obtained may be used as an additive in detergents and cleaning agents.

The thickened perfume compositions of the invention are used as additive in laundry, cleaning and fabric care compositions and in softeners. Such compositions usually contain, for example, of from 0.0001% to 10% by weight, preferably from 0.0001 to 5% by weight and more preferably from 0.01 to 2% by weight of the thickened perfume composition.

The viscosity of the compounds was measured in a Brookfield viscometer at 20 rpm and at 20° C. The molecular weight of the polymers means the weight average molecular weight M_{w} which was measured by gel-permeation-chromatography (GPC). The following commercially available products were used:

Perfume oil 1 having the following compositions:

| Perfume Ingredients | Wt. % |
|---|---|
| Benzophenone | 3 |
| Benzylacetate | 10 |
| Benzylsalicylate | 5 |
| Cedrol | 2 |
| Citronellol | 10 |
| Dihydromyrcenol | 10 |
| Floracetate | 5 |
| Galaxolide | 10 |
| Lilial | 10 |
| Linalylacetate | 4 |
| Linalool | 6 |
| Methyldihydrojasmonate | 3 |
| Phenylethylacetate | 2 |
| Phenylethylalcohol | 15 |

Luvotix® HT is a hydrogenated castor oil (Rheox).

Thixatrol® ST is a hydrogenated castor oil (Rheox).

A rosilo® 380 is fumed silica (Degussa AG, Frankfurt).

### COMPARATIVE EXAMPLE 1

80 g of a polyethyleneimine with molecular weight M_{w} 25,000 were heated to 60° C. As soon as this temperature was reached a mixture of 120 g δ-Damascone und 200 g perfume oil 1 were added. The mixture was then stirred for 30 minutes at 60° C. and cooled to room temperature to yield an orange, solution with a viscosity of 2300 mPas.

### EXAMPLE 1

70 g of the product obtained in Comparative Example 1 were heated to 80° C. At this temperature 1.4 g of ethyleneglycol diglycidyl-ether were added dropwise over a 5-minute period while stirring. Stirring was continued for 3 h at 80° C. Upon cooling to room temperature a viscous yellow oil with a viscosity or 16,600 mPas was obtained.

### EXAMPLE 2

70 g of the product obtained in Comparative Example 1 were heated to 80° C. At this temperature 1.54 g ethyleneglycol diglycidyl-ether were added dropwise over a period of 5 minutes while stirring. The stirring was continued for 3 h at 80° C. Upon sling to room temperature a viscous yellow oil with a viscosity of 29760 mPas was obtained.

### EXAMPLE 3

3.5 g of Aerosil 380 ware added to 70 g of the product obtained in Comparative Example 1 while stirring at room temperature. The stirring was continued for 3 h. A viscous yellow oil having a viscosity of 13100 mPas was obtained.

### EXAMPLE 4

4.9 g Aerosil 380 were added to 70 g of the product obtained in Comparative Example 1 while stirring at room temperature. Stirring was continued for 3 h. A viscous yellow oil having a viscosity of 25680 mPas was obtained.

### EXAMPLE 5

1.2 g Luvotix HT were added to 60 g of the product obtained in Comparative Example 1 at room temperature while stirring. The mixture was then heated to 80° C. and stirred for 15 minutes at this temperature and then cooled to room temperature. A yellow pasty material was obtained.

### EXAMPLE 6

1.2 g Thixatrol ST were added to 60 g of the product obtained in Comparative Example 1 while stirring at room temperature. The mixture was then heated to 80° C., stirred for 15 minutes at this temperature and then cooled to room temperature. A yellow pasty material was obtained.

### COMPARATIVE EXAMPLE 2

20 g of a polyvinylamine with molecular weight of 6,000 were heated to 80° C. As soon as this temperature was reached, a mixture of 30 g of 6-Damascone and 75 g of perfume oil 1 were added. The mixture was then stirred for 4 h at 80° C. and cooled to room temperature to yield a viscous yellow oil with a viscosity of 23,480 mPas.

### EXAMPLE 7

30 g of the product obtained in Comparative Example 2 were heated to 80° C. At this temperature 0.3 g of ethyleneglycol diglycidyl-ether were added dropwise aver a 5-minute period while stirring. Stirring was continued for 3 h at 80° C. Upon cooling to room temperature a viscous yellow oil with a viscosity of 36,100 mPas was obtained.

### EXAMPLE 8

0.5 g Luvotix HT were added to 25 g of the product obtained in Comparative Example 2 at room temperature while stirring. The mixture was then heated to 80° C. and stirred for 30 minutes at this temperature and the cooled to room temperature.

A viscous yellow oil with a viscosity of 47,000 mPas was obtained.

### EXAMPLE 9

1.5 g Luvotix HT were added to 30 g of the product obtained in Comparative Example 2 at room temperature while stirring. The mixture was then heated to 80° C. and stirred for 30 minutes at this temperature and then cooled to room temperature. A viscous yellow oil with a viscosity of 230,000 mPas was obtained.

### COMPARATIVE EXAMPLE 3

20 g of a polylysine with molecular weight 14,000 were heated to 80° C. As soon as this temperature was reached, a mixture of 30 g of δ-Damascone and 50 g perfume oil 1 were added. The mixture was then stirred for 2 h at 80° C. and cooled to room temperature to yield a yellow oil with a viscosity of 530 mPas.

### EXAMPLE 10

25 g of the product obtained in Comparative Example 3 were heated to 80° C. At this temperature 0.5 g of ethyleneglycol diglycidyl-ether were added dropwise over a 5-minute period while stirring. Stirring was continued for 3 h at 80° C. Upon cooling to room temperature a yellow oil with a viscosity of 1,372 mPas was obtained.

### EXAMPLE 11

25 g of the product obtained in Comparative Example 3 were heated to 80° C. At this temperature 1 g of ethyleneglycol diglycidyl-ether were added dropwise over a 5-minute period while stirring. Stirring was continued for 3 h at 80° C. Upon cooling to room temperature a viscous yellow oil with a viscosity of 4,320 mPas was obtained.

### EXAMPLE 12

0.18 g Luvotix UT were added to 18 g of the product obtained in Comparative Example 3 at room temperature while stirring. The mixture was then heated to 80° C. and stirred for 15 minutes at this temperature and then cooled to room temperature.

A yellow oil with a viscosity of 1.320 mPas was obtained.

### EXAMPLE 13

0.5 g Luvotix HT were added to 25 g of the product obtained in Comparative Example 3 at room temperature while stirring. The mixture was then heated to 80° C. and stirred for 19 minutes at this temperature and then cooled to room temperature.

A pasty material wan obtained.

1. A perfume composition which is obtainable by adding to 100 parts by weight of a mixture of
(a) 10 to 95% by weight of at least one perfume compound and
(b) 5 to 90% by weight of at least one polyamine,
the sum of (a) and (b) being always 100%, 0.1 to 20 parts by weight of at least one crosslinking agent having at least two groups which react with primary or secondary amino groups of the polyamine and crosslinking the mixture, and/or adding 0.1 to 30 parts by weight of a thickening agent.

2. A perfume composition as claimed in claim 1, wherein the perfume compound is selected from the group consisting of from α-damascone, δ-damascone, iso-damascone, carvone, γ-methyl-lonone, 2,4,4,7-tetramethyl-oct-6-en-3-one, benzyl acetone, β-damascone, damascone, methyl dihydrojasmonate, methyl cedrylone, and mixtures thereof.

3. A perfume composition as claimed in claim 1, wherein the perfume compound is selected from 1-decanal, benzaldehyde, florhydral, 2,4-dimethyl-3-cyclohexene-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, bucinal, lyral, cymal, methyl nonyl acetaldehyde, hexanal, trans-2-hexenal, and mixtures thereof.

4. A perfume composition as claimed in any of claims 1 to 3, wherein the polyamine is selected from the group consisting of polymers containing vinylamine units, polyethyleneimines, polymers grafted with ethyleneimine, polyallylamines, condensation products of piperazine, 1-(2-aminoethyl)piperazine, 1,4-bis(3-aminopropyl)piperazine and mixtures thereof with crosslinkers, polymers containing lysine units, dendrimers containing primary amino groups, and mixtures thereof.

5. A perfume composition as claimed in any of claims 1 to 4, wherein the polyamine is polyethyleneimine having a molecular weight of from 600 to 200,000.

6. A perfume composition as claimed in any of claims 1 to 5, wherein the crosslinking agent is selected from the group consisting of epichlorohydrin, bischlorohydrin ethers of ethylene glycol, polyethylene glycol having 2 to 100 glycol units, propylene glycols, polypropylene glycols, copolymers of ethylene oxide and propylene oxide, glycerol, diglycerol, polyglycerol having up to 8 glycerol units, pentaerythritol and sorbitol, epoxides obtained from said bischlorohydrin ethers and mixtures thereof.

7. A perfume composition as claimed in any of claims 1 to 6, wherein the crosslinking agent is a diglycidyl ether.

8. A perfume composition as claimed in any of claims 1 to 7, wherein the thickener is selected from the group consisting of hydrogenated castor oil, fumed silica or bentonite.

9. A process for the production of a perfume composition which comprises adding to 100 parts by weight of a mixture of
(a) 10 to 95% by weight of at least one perfume compound and
(b) 5 to 90% by weight of at least one polyamine,
the sum of (a) and (b) being always 100%,
(c) 0.1 to 20 parts by weight of at least one crosslinking agent having at least two groups which react with primary or secondary amino groups of the polyamine and crosslinking the mixture, and/or adding 0.1 to 30 parts by weight of a thickening agent.

10. A process as claimed in claim 9, wherein the polyamine is selected from the group consisting of polymers containing vinylamine units, polyethyleneimines, polymers grafted with ethyleneimine, polyallylamines, condensation products of piperazine, 1-(2-aminoethyl)piperazine, 1,4-bis(3-aminopropyl) piperazine and mixtures thereof with crosslinkers, polymers containing lysine units, dendrimers containing primary amino groups, and mixtures thereof.

11. A process an claimed in claim 11 or 12, wherein the polyamine is selected from the group consisting of polyvinylamine, a copolymer containing vinylamine units, their salts with inorganic or organic acids, and mixtures thereof.

12. A process as claimed in any of claims 9 to 11, wherein the crosslinking agent is selected from the group consisting of epichlorohydrin, bischlorohydrin ethers of ethylene glycol, polyethylene glycols having 2 to 100 glycol units, propylene glycols, polypropylene glycols, copolymers of ethylene oxide and propylene oxide, glycerol, diglycerol, polyglycerol having up to 8 glycerol units, pentaerythritol and sorbitol, glycidyl ethers obtained from said bischlorohydrin ethers, and mixtures thereof.

13. A process as claimed in any of claims 9 to 12, wherein the crosslinking agent is a diglycidyl ether of ethylene glycol or polyethylene glycol having 2 to 50 ethylene glycol units.

14. A process as claimed in any of claims 9 to 13, wherein the thickener is selected from the group consisting of hydrogenated castor oil, fumed slica, bentonite and mixtures thereof.

15. Use of the perfume composition as claimed in claims 1 to 8 as additive in laundry, cleaning and fabric care compositions and in softeners.

16. Cleaning composition comprising the perfume composition an claimed in claims 1 to 8.

17. Fabric care composition comprising the perfume composition as claimed in claims 1 to 8.

18. Cleaning composition or fabric care composition comprising the perfume composition according to any preceeding claim and a softening agent.
